(19) United States
(12) Patent Application Publication
(10) Pub. No.: US 2011/0034418 A1
(43) Pub. Date: Feb. 10, 2011

(54) PHARMACEUTICAL COMPOSITION WITH BISPHOSPHONATE

(76) Inventors: Karen Beltz, Basel (CH); Philipp Lustenberger, Basel (CH); Holger Petersen, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/935,402

(22) PCT Filed: Apr. 2, 2009
(86) PCT No.: PCT/EP09/53965

§ 371 Date: Sep. 29, 2010

(30) Foreign Application Priority Data
Apr. 4, 2008 (EP) 08154114.6

(51) Int. Cl. A61K 31/675; C07F 9/28; A61P 19/08

(52) U.S. Cl. 514/94; 548/112

(57) ABSTRACT

The present invention relates to depot formulations comprising a poorly water soluble salt of a bisphosphonate forming together with one or more biocompatible polymers, to poorly water-soluble salts of such bisphosphonates, to crystalline forms of the free compounds and the salts and to other related aspects, where the compounds are of the Formula (I), where R₁ and R₂ are as described in the specification. Compounds of the Formula (I) and their forms mentioned in the disclosure are useful for the treatment of bone-related disorders and cancer.

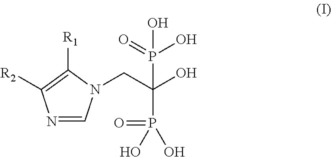

### FIELD OF THE INVENTION

The present invention relates to depot formulations comprising a poorly water soluble salt (also referred to as poorly soluble salt hereinafter, meaning poorly water soluble) of a bisphosphonate forming together with one or more biocompatible polymers. The depot formulation may be in the form of microparticles or implants. The depot formulations are useful for the treatment and prevention of various, e.g. bone related and/or proliferative, diseases, especially degenerative diseases and rheumatoid arthritis and osteoarthritis.

In a further aspect, the present invention relates to new salts including new crystal forms of said salts of certain bisphosphonates, as well as new crystal forms of the bisphosphonates in free (e.g. zwitterionic) form.

Further, various other embodiments (uses, methods, processes or methods for preparation and related subject matter) are embodiments of the invention.

### BACKGROUND OF THE INVENTION

Bisphosphonates are widely used to inhibit osteoclast activity in a variety of both benign and malignant diseases in which bone resorption is increased. So far, only water soluble bisphosphonates, e.g., the sodium salt, have been used in pharmaceutical compositions. In case of forming solutions for infusion this is a reasonable approach. However, in case of a depot formulation the high water solubility of the bisphosphanate will lead to a high initial release causing severe local tissue irritations.

For example, the drug zoledronic acid is used in the prevention of skeleton related events, such as, inter alia, pathological fractures, spinal compression, radiation or surgery to bone or tumor-induced hypercalcemia) in patients with various diseases or disorders e.g. involving bone and calcium metabolism, such as advanced malignancies involving bone, treatment of tumor-induced hypercalcemia, Paget's disease, operation and prevention of hip fractures, or the like.

### OVERVIEW OVER THE INVENTION

It has now been surprisingly found that poorly water soluble bisphosphonates of a novel class of bisphosphonates can be encapsulated very efficiently so that the drug release is very well under control.

One advantage of a poorly water soluble salt is that generally the encapsulation of the drug substance is improved because highly water soluble salts may dissolve into the aqueous phase during the manufacturing of the microparticles via commonly used emulsion-solvent evaporation/extraction method. A further advantage is that the drug release out of the resulting depot formulation is generally better controlled if the drug substance has limited water solubility compared to highly water soluble salts.

An advantage of the micronization of the drug substance is the more complete encapsulation of the drug substance particles in polymer matrices compared to large drug substance particles which may only partly been encapsulated in the matrix leading to an uncontrolled release of the drug substance.

In addition, new salts have been found that enable the manufacture of the aforementioned formulations.

Further, new crystal forms of certain bisphosphonates and their salts (including hydrates or other solvates) have been found and are an embodiment of the invention.

### DETAILED DESCRIPTION OF THE DRAWINGS

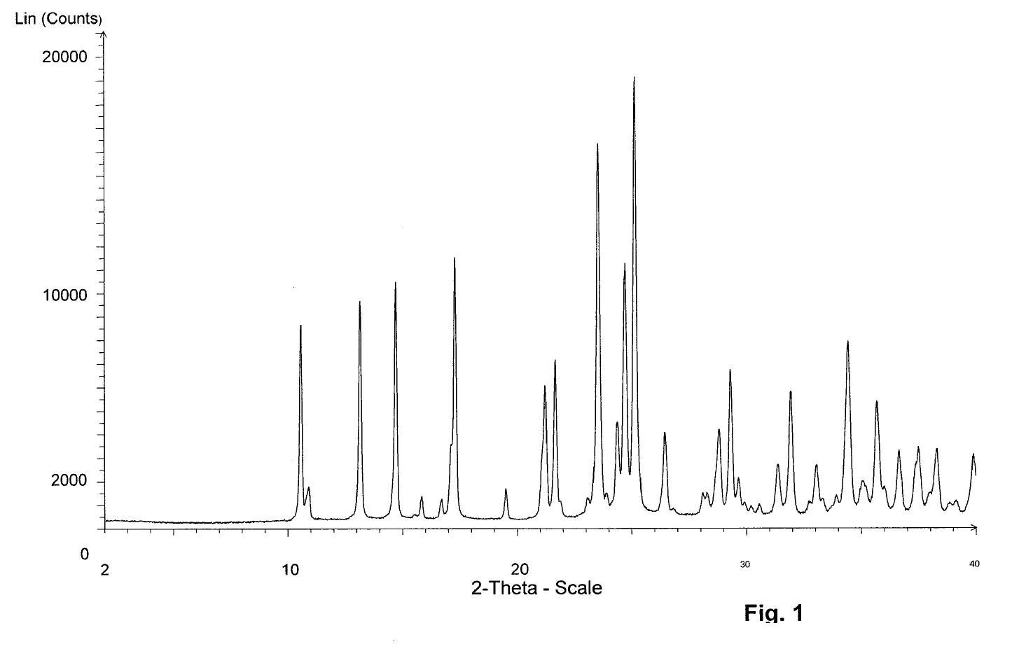

FIG. 1 shows the X-ray diffractogram of the crystalline zwitterionic (internal) salt of [2-(5-ethyl-imidazol-1-yl)-1-hydroxy-1-phosphono-ethyl]-phosphonic acid (Cpd. A), for details see Example 6.

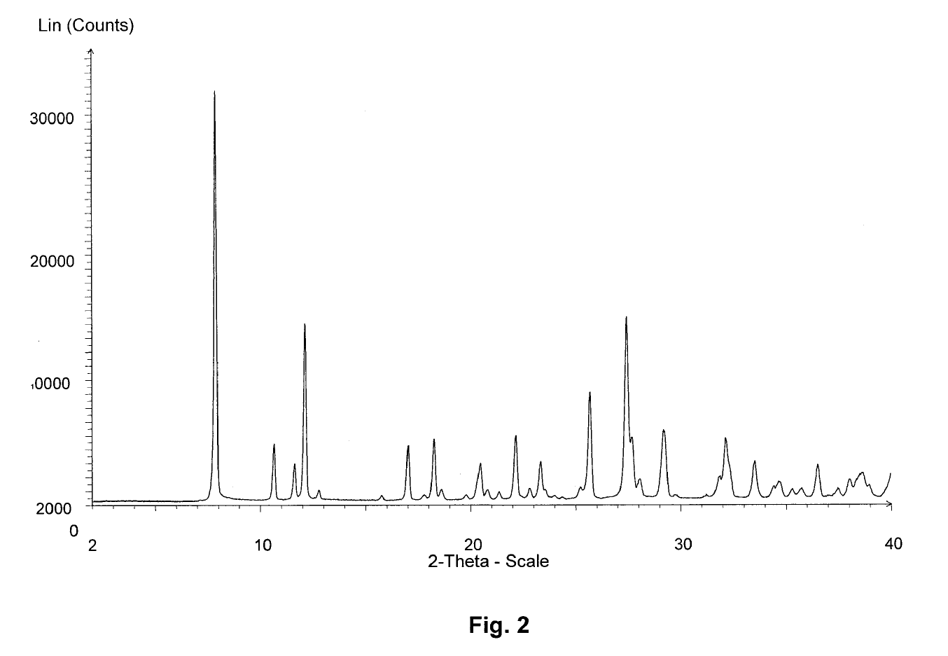

FIG. 2 shows the X-ray diffractogram of the crystalline Ca-salt of Cpd. A (1:2), for details see Example 7.

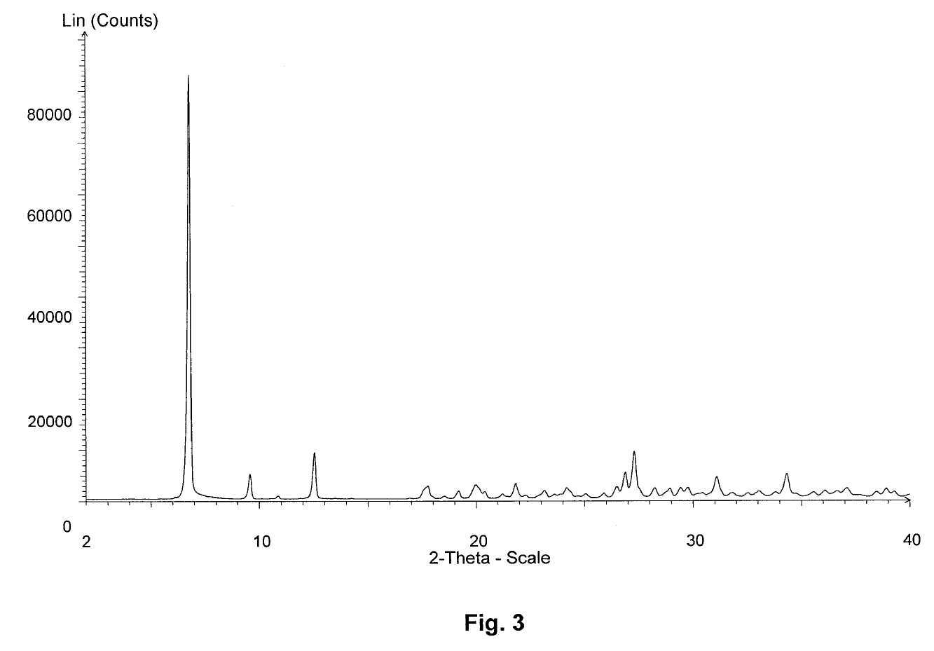

FIG. 3 shows the X-ray diffractogram of the crystalline Mg-salt of Cpd. A (1:2), for details see Example 8.

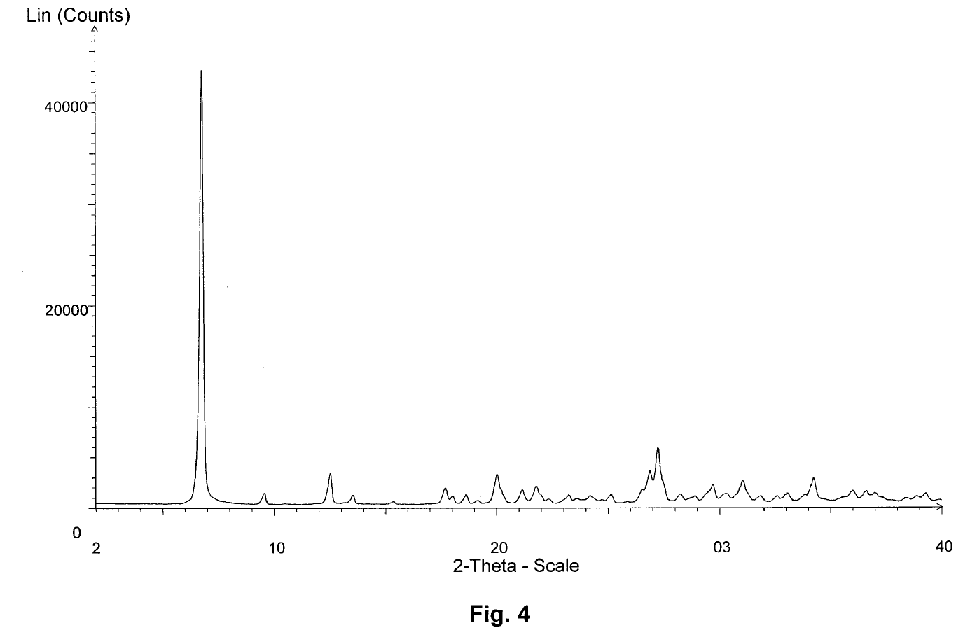

FIG. 4 shows the X-ray diffractogram of the crystalline Zn-salt of Cpd. A (1:2), for details see Example 9.

### DETAILED DESCRIPTION OF THE INVENTION

In the following detailed description of the invention, more specific definitions provided for general terms in one of the embodiments may also be used to define the general terms more specifically in other embodiment, this forming more specific embodiments of the invention, with the proviso, that each term may be replaced independently of the other general terms defining an embodiment of the invention.

The present invention, in a first embodiment, relates to depot formulations comprising a poorly water soluble salt of a bisphosphonate of the formula I together with biocompatible polymers.

The present invention in this regard especially relates to depot formulations comprising a poorly water soluble salt of a bisphosphonate forming together with one or more biocompatible polymers, where the bisphosphonate compound is a compound selected from compounds of the formula I,

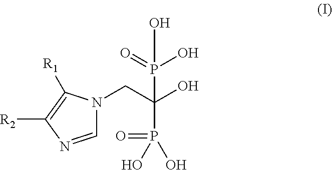

wherein one of R₁ and R₂ is hydrogen and the other is C₁-C₅-alkyl (preferably C₂-C₅-alkyl) that is branched or unbranched in the form of a poorly water-soluble salt.

Preferred is a depot formulation of the bisphosphonate of the formula I, wherein one of R₁ and R₂ is hydrogen and the other is methyl in the form of a poorly water-soluble salt. Alternatively, a depot formulation of the bisphosphonate of the formula I wherein one of R₁ and R₂ is hydrogen and the other is ethyl in the form of a poorly water-soluble salt is very preferred.

Most preferred is a depot formulation of the bisphosphonate of the formula I with the name [2-(5-methyl-imidazol-1-yl)-1-hydroxy-1-phosphono-ethyl]-phosphonic acid or more preferably [2-(5-ethyl-imidazol-1-yl)-1-hydroxy-1-phosphono-ethyl]-phosphonic acid in the form of a poorly water-soluble salt.

Also the poorly water-soluble salts of compounds of the formula I, especially the salts with the preferred compounds of the formula I as defined in the preceding paragraphs, as such are an embodiment of the invention, especially in the form of specific polymorphs (crystal forms or crystal modifications) as described below in more detail.

“Poorly soluble”, wherever used in this text, means that the solubility is 2 mg/ml in water at a temperature from 21 to 24° C., more preferably less than 1 mg/ml of water at said temperature.

The present invention relates especially to depot formulations in the form of microparticles comprising a poorly water soluble salt of a bisphosphonate of the formula I together with one or preferably more biocompatible polymers, preferably biodegradable polymers.

The present invention also relates to implants comprising a poorly water soluble salt of a bisphosphonate of the formula I together with one or preferably more biocompatible polymers, preferably biodegradable polymers.

The present invention relates to methods for the treatment and prevention of diseases or disorders where abnormal bone turnover is found, as provided in more detail below, comprising administering a depot formulation or a poorly soluble salt or a crystalline form of a free form (or its internal salt, e.g. zwitterionic salt) of a compound of the formula Ito a patient in need of such treatment in a therapeutically effective dosage, as well as the use of a depot formulation or poorly soluble salt or a crystalline form of a free form (or its internal salt, e.g. zwitterionic salt) of a compound of the formula I in the manufacture of medicaments for the treatment of such diseases or disorders and their use in the treatment of said disorders or diseases, as well as the depot formulations or salts a crystalline forms of a free form (or its internal salt, e.g. zwitterionic salt) of a compound of the formula I for use in such treatment.

The poorly water-soluble salt of a compound of the formula I which is an embodiment of the invention or is part of a depot formulation according to the invention is selected from the calcium, magnesium and zinc salt, or a mixture of two or all of these salts, preferably as 1:1 or especially 1:2 salts (herein wherever mentioned giving the molar ratio of (metal ion):(compound of the formula I), where “metal” refers to calcium, magnesium and/or (especially “or”) zinc). These salts are low in water solubility, in other terms, poorly water soluble means that the water solubility is 25% or less of a corresponding sodium salt.

Preferably, the depot formulations of the invention contain as active ingredient only a compound of formula I, preferably [2-(5-methyl-imidazol-1-yl)-1-hydroxy-1-phosphono-ethyl]-phosphonic acid or especially [2-(5-ethyl-imidazol-1-yl)-1-hydroxy-1-phosphono-ethyl]-phosphonic acid, in the form of its poorly water soluble salt, or a crystalline form of a compound of the formula I named [2-(5-ethyl-imidazol-1-yl)-1-hydroxy-1-phosphono-ethyl]-phosphonic acid in free (e.g. especially zwitterionic) form.

It has been found that the calcium salts are better polymer-encapsulated in the formulations according to the invention than the zinc salts—therefore, calcium salts of a compound of the formula I are generally more preferred, especially for the depot formulations.

In addition, the defined crystal forms both of the free compounds as well as the salts of the compounds of the formula I, respectively, show additional advantages, e.g. a fixed stoichiometric relationship between their components and, where solvates, such as hydrates, are formed, the solvent molecules, good millability to yield particles in the micrometer range, good flowability and other advantageous properties of crystalline over amorphic materials that facilitate the processing of such materials to pharmaceutical formulations, also including improved storability.

Preferably, the microparticles of the invention contain a compound of formula I, in form of the calcium salt, even more preferably the calcium salt of [2-(5-ethyl-imidazol-1-yl)-1-hydroxy-1-phosphono-ethyl]-phosphonic acid.

The bisphosphonates of the formula I may be present in an amount of from about 1% to about 60%, more usually about 2% to about 20%, preferably about 5% to about 10%, by weight of the depot dry weight of the microparticle formulation.

The bisphosphonates of the invention are released from the depot formulations of the invention and from the compositions of the invention over a period of several weeks, e.g., about 2 weeks to 18 months, e.g. from 3 weeks to 12 months.

Preferably, the bisphosphonate of the formula I in the form of its poorly water-soluble salt used to prepare the depot formulations is a very fine powder produced by any type of micronization technique (e.g., jet milling or high pressure homogenization) having a particle size (e.g. with 90% of the weight of the particles in that range, preferably 98%) of about 0.1 microns to about 15 microns, preferably less than about 5 microns, even more preferably less than about 3 microns. It is found that micronizing the drug substance improves the encapsulation efficiency.

In accordance with one aspect, the invention a calcium salt of a compound of the formula I, especially with a stoichiometry of one calcium and two molecules of compound of the formula I (an 1:2 salt), especially a calcium salt of Compound A (=Cpd. A=[2-(5-ethyl-imidazol-1-yl)-1-hydroxy-1-phosphono-ethyl]-phosphonic acid).

In accordance with another aspect, the invention provides a zinc salt of a compound of the formula I, especially with a stoichiometry of one zinc and two molecules of compound of the formula I (an 1:2 salt) or of one zinc and two molecules of compound of the formula I (an 1:1 salt), especially a zinc salt of Compound A (=Cpd. A=[2-(5-ethyl-imidazol-1-yl)-1-hydroxy-1-phosphono-ethyl]-phosphonic acid).

In accordance with yet another aspect, the invention provides a magnesium salt of a compound of the formula I, especially with a stoichiometry of one Magnesium and two molecules of compound of the formula I (a 1:2 salt), especially a magnesium salt of Compound A (=Cpd. A=[2-(5-ethyl-imidazol-1-yl)-1-hydroxy-1-phosphono-ethyl]-phosphonic acid).

Moreover, it has surprisingly been found that the compounds of the formula I in free form (this term always including internal salts, such as zwitterionic forms) as well as salts of compounds of the formula I can be present in polymorphic forms (different crystal modifications).

The invention therefore in a further embodiment relates to new crystalline forms of low water soluble salts of compounds of the formula I or their free (e.g. zwitterionic) form, especially of [2-(5-ethyl-imidazol-1-yl)-1-hydroxy-1-phosphono-ethyl]-phosphonic acid (Cpd. A hereinafter), the process for preparation of these crystalline forms, compositions containing these crystalline forms, and the use of these crystalline forms in diagnostic methods or therapeutic treatment of warm-blooded animals, especially humans.

Both the free forms as well as the salt forms, each in crystalline form, may be free of solvent or (especially in the case of the salts) in solvate, e.g. hydrate form, e.g. as the dihydrate.

With regard to the crystalline forms, the invention, in a first aspect, provides a crystalline form of the free form or one of the salt forms (especially a salt in hydrate form) of a compound of the formula I.

In a more focused aspect, the invention provides a crystalline form of the free zwitterionic form of Cpd. A, which more preferably has an X-ray powder diffraction pattern with at least one, preferably two, more preferably three, most preferably all of the following peaks at an angle of refraction 2 theta (θ) of 10.5, 13.1, 14.7, 17.2, 23.5, 25.2 and 29.2, ±0.2, respectively, especially as depicted in FIG. 1; alternatively, at least 80% by weight of Cpd. A in the free zwitterionic form shows such X-ray powder diffraction pattern.

In another more focused aspect, the invention provides a crystalline form of the calcium salt of Cpd. A (especially in the hydrate form, such as the dihydrate) with a stoichiometry of one calcium and two molecules of Cpd. A, which more preferably has an X-ray powder diffraction pattern with at least one, preferably two, more preferably three, most preferably all of the following peaks at an angle of refraction 2 theta (θ) of 7.9, 10.6, 12.1, 25.7, 27.4 and 29.2, ±0.2, respectively, especially as depicted in FIG. 2; alternatively, at least 80% by weight of the calcium 1:2 salt of Cpd. A shows such X-ray powder diffraction pattern.

In yet another more focused aspect, the invention provides a crystalline form of the zinc salt of Cpd. A (especially in the hydrate form, such as the dihydrate) with a stoichiometry of one zinc and two molecules of Cpd. A, which more preferably has an X-ray powder diffraction pattern with at least one, preferably two, more preferably three, most preferably all of the following peaks at an angle of refraction 2 theta (θ) of 6.7, 9.5, 12.5, 17.7 and 27.3, ±0.2, respectively, especially as depicted in FIG. 3; alternatively, at least 80% by weight of the zinc 1:2 salt of Cpd. A shows such X-ray powder diffraction pattern.

In yet another more focused aspect, the invention provides a crystalline form of the magnesium salt of Cpd. A (especially in the hydrate form, such as the dihydrate) with a stoichiometry of one magnesium and two molecules of Cpd. A, which more preferably has an X-ray powder diffraction pattern with at least one, preferably two, more preferably three, most preferably all of the following peaks at an angle of refraction 2 theta (θ) of 6.7, 12.5, 20.0 and 27.3, ±0.2, respectively, especially as depicted in FIG. 4; alternatively, at least 80% by weight of the magnesium 1:2 salt of Cpd. A shows such X-ray powder diffraction pattern.

The parameters and devices for retrieval of the X-ray data mentioned above and in the claims preferably are in accordance with those mentioned below in the Examples.

In accordance with another aspect of the invention, the invention provides a pharmaceutical formulation (especially a depot formulation as herein described) including a crystalline form, especially as described in any one of the abovementioned focused aspects of the invention, of a compound of the formula I or a poorly soluble salt thereof, especially a calcium salt (calcium: Cpd. A=1:2 being especially preferred, especially in the hydrate, e.g. dihydrate, form) and at least one pharmaceutically acceptable carrier, especially for parenteral administration.

In yet another aspect, the invention relates to an amorphous or crystalline form of a compound of the formula I, especially Cpd. A, in the form of a poorly soluble salt selected from the zinc, (especially) magnesium and (more especially) calcium salt, especially where the stoichiometry of the metal ion to the compound of the formula I is 1:2; or to a crystalline form of a compound of the formula I, especially in its free (e.g. inner zwitterionic) form or in the form of an (especially 1:1 or more especially 1:2) zinc, (especially) magnesium or (more especially) calcium salt, each especially in hydrate form, e.g. in the form of a dihydrate, or a mixture of two or more such forms, especially for use in the treatment of one or more diseases or disorders where abnormal bone turnover is found (the term treatment wherever used in this disclosure including both prophylactic and therapeutic (e.g. palliative or curing) treatment.

About, where used in this specification, especially means that the number mentioned after “about” can vary by plus 10 to minus 10 percent of its absolute value.

The particle size distribution of the poorly water-soluble salts of bisphosphonates of the formula I may influence the release profile of the drug. Typically, the smaller the particle size, the lower is the burst and release during the first diffusion phase, e.g., the first 20 days. Preferably, particle size distribution is, e.g., ×10 <2 microns, i.e., 10% of the particles are smaller than 2 microns; ×50<5 microns, i.e., 50% of the particles are smaller than 5 microns; or ×90<10 microns, i.e., 90% of the particles are smaller than 10 microns.

### II. Microparticles

It has been found that administration of microparticles comprising a low soluble salt of a bisphosphonate of the formula I embedded in a biocompatible pharmacologically acceptable polymer, preferably a biodegradable pharmacologically acceptable polymer, suspended in a suitable vehicle gives release of the active agent over an extended period of time, e.g., one week up to 18 months, preferably for about 3 weeks to about 12 months.

The present invention in another aspect provides a process for the preparation of microparticles of the invention comprising:

(i) preparation of an internal organic phase comprising:
(ia) dissolving the polymer or polymers in a suitable organic solvent or solvent mixture, and optionally dissolving/dispersing a porosity-influencing agent in the solution obtained in step (ia), or
   adding a basic salt to the solution obtained in step (ia),
   adding a surfactant to the solution obtained by step (ia);
(ib) suspending a poorly water-soluble salt of a compound of the formula I in the polymer solution obtained in step (ia), or dissolving a poorly water-soluble salt of a compound of the formula I in a solvent miscible with the solvent used in step (ia) and mixing said solution with the polymer solution, or directly dissolving a poorly water-soluble salt of a compound of the formula I in the polymer solution;

(ii) preparation of an external aqueous phase comprising
(iia) preparing a buffer to adjust the pH to 3.0-8.0, for example pH 3.0-5.0; e.g., acetate buffer, and
(iib) dissolving a stabilizer in the solution obtained in step (iia);

(iii) mixing the internal organic phase with the external aqueous phase, e.g., with a device creating high shear forces, e.g., with a turbine or static mixer, to form an emulsion; and

(iv) hardening the microparticles by solvent evaporation or solvent extraction, optionally in addition washing the microparticles, e.g., with water, and collecting and drying the microparticles, e.g., by freeze-drying or drying under vacuum.

Suitable organic solvents for the polymers include, e.g., ethyl acetate or halogenated hydrocarbons, e.g., methylene chloride, chloroform, or mixtures of two or more e.g. of them.

Suitable examples of a stabilizer for step (iib) include:
a) Polyvinyl alcohol (PVA), preferably having a weight average molecular weight from about 10,000 Da to about 150,000 Da, e.g., about 30,000 Da. Conveniently, the polyvinyl alcohol has low viscosity having a dynamic viscosity of from about 3 mPa s to about 9 mPa s when measured as a 4% aqueous solution at 20° C. or by DIN 53015. Suitably, the polyvinyl alcohol may be obtained from hydrolyzing polyvinyl acetate. Preferably, the content of the polyvinyl acetate is from about 10% to about 90% of the polyvinyl alcohol. Conveniently, the degree of hydrolysis is about 85% to about 89%. Typically the residual acetyl content is about 10-12%. Preferred brands include Mowiol® 4-88, 8-88 and 18-88 available from Kuraray Specialities Europe, GmbH.
   Preferably the polyvinyl alcohol is present in an amount of from about 0.1% to about 5%, e.g., about 0.5%, by weight of the volume of the external aqueous phase;
b) Hydroxyethyl cellulose (HEC) and/or hydroxypropyl cellulose (HPC), e.g., formed by reaction of cellulose with ethylene oxide and propylene oxide, respectively. HEC and HPC are available in a wide range of viscosity types; preferably the viscosity is medium. Preferred brands include Natrosol® from Hercules Inc., e.g., Natrosol® 250MR and Klucel® from Hercules Inc.
   Preferably, HEC and/or HPC is present in a total amount of from about 0.01% to about 5%, e.g., about 0.5%, by weight of the volume of the external aqueous phase;
c) Polyvinylpyrolidone, e.g., suitably with a molecular weight of between about 2,000 Da and 20,000 Da. Suitable examples include those commonly known as Povidone K12 F with an average molecular weight of about 2,500 Da, Povidone K15 with an average molecular weight of about 8,000 Da, or Povidone K17 with an average molecular weight of about 10,000 Da. Preferably, the polyvinylpyrolidone is present in an amount of from about 0.1% to about 50%, e.g., 10% by weight of the volume of the external aqueous phase
d) Gelatin, preferably porcine or fish gelatin. Conveniently, the gelatin has a viscosity of about 25 cps to about 35 cps for a 10% solution at 20° C. Typically pH of a 10% solution is from about 6 to about 7. A suitable brand has a high molecular weight, e.g., Norland high molecular weight fish gelatin obtainable from Norland Products Inc, Cranbury, N.J., USA.
   Preferably, the gelatin is present in an amount of from about 0.01% to about 5%, e.g., about 0.5%, by weight of the volume of the external aqueous phase.
   Preferably, polyvinyl alcohol is used. Preferably, no gelatin is used. Preferably, the microparticles are gelatin-free.

The resulting microparticles may have a diameter from a few submicrons to a few millimeters; e.g., diameters of at most, e.g., 5-200 microns, preferably 5-130 microns, more preferably 5-100 microns are strived for, e.g., in order to facilitate passage through an injection needle. A narrow particle size distribution is preferred. For example, the particle size distribution may be, e.g., 10%<20 microns, 50%<50 microns or 90%<80 microns.

Content uniformity of the microparticles and of a unit dose is excellent. Unit doses may be produced which vary from about 20% to about 125%, e.g., about 70% to about 115%, e.g., from about 90% to about 110%, or from about 95% to about 105%, of the theoretical dose.

The microparticles in dry state may, e.g., be mixed, e.g., coated, with an anti-agglomerating agent, or, e.g., covered by a layer of an anti-agglomerating agent, e.g., in a prefilled syringe or vial.

Suitable anti-agglomerating agents include, e.g., mannitol, glucose, dextrose, sucrose, sodium chloride or water soluble polymers, such as polyvinylpyrrolidone or polyethylene glycol, e.g., with the properties described above.

Preferably, an anti-agglomerating agent is present in an amount of about 0.1% to about 10%, e.g., about 4% by weight of the microparticles.

Prior to (usually s.c. or i.m.) administration, the microparticles are suspended in a vehicle suitable for injection.

Accordingly, the present invention further provides a pharmaceutical composition comprising microparticles of the invention in a vehicle. The vehicle may optionally further contain:
a) one or more wetting agents; and/or
b) one or more tonicity agent; and/or
c) one or more viscosity increasing agents.

Preferably, the vehicle is water based, e.g., it may contain water, e.g., deionized, and optionally a buffer to adjust the pH to 7-7,5, e.g., a phosphate buffer, such as a mixture of Na₂HPO₄ and KH₂PO₄, and one or more of agents a), b) and/or c) as indicated above.

However, when using water as a vehicle, the microparticles of the invention may not suspend and may float on the top of the aqueous phase. In order to improve the capacity of the microparticles of the invention to be suspended in an aqueous medium, the vehicle preferably comprises a wetting agent a). The wetting agent is chosen to allow a quick and suitable suspendibility of the microparticles in the vehicle. Preferably, the microparticles are quickly wettened by the vehicle and quickly form a suspension therein.

Suitable wetting agents for suspending the microparticles of the invention in a water-based vehicle include non-ionic surfactants, such as poloxamers, or polyoxyethylene-sorbitan-fatty acid esters, the characteristics of which have been described above. A mixture of wetting agents may be used. Preferably, the wetting agent comprises Pluronic F68, Tween 20 and/or Tween 80.

The wetting agent or agents may be present in about 0.01% to about 1% by weight of the composition to be administered, preferably from 0.01-0.5% and may be present in about 0.01-5 mg/mL of the vehicle, e.g., about 2 mg/mL.

Preferably, the vehicle further comprises a tonicity agent b), such as mannitol, sodium chloride, glucose, dextrose, sucrose or glycerin. Preferably, the tonicity agent is mannitol.

The amount of tonicity agent is chosen to adjust the isotonicity of the composition to be administered. In case a tonicity agent is contained in the microparticles, e.g., to reduce agglomeration as mentioned above, the amount of tonicity agent is to be understood as the sum of both. For example, mannitol preferably may be from about 1% to about 5% by weight of the composition to be administered, preferably about 4.5%.

Preferably, the vehicle further comprises a viscosity increasing agent c). Suitable viscosity increasing agents include carboxymethyl cellulose sodium (CMC-Na), sorbitol, polyvinylpyrrolidone, or aluminum monostearate.

CMC-Na with a low viscosity may conveniently be used. Embodiments may be as described above. Typically, a CMC-Na with a low molecular weight is used. The viscosity may be of from about 1 mPa s to about 30 mPa s, e.g., from about 10 mPa s to about 15 mPa s when measured as a 1% (w/v) aqueous solution at 25° C. in a Brookfield LVT viscometer with a spindle 1 at 60 rpm, or a viscosity of 1-15 mPa*s for a solution of NaCMC 7LF (low molecular weight) as a 0.1-1% solution in water.

A polyvinylpyrrolidone having properties as described above may be used.

A viscosity increasing agent, e.g., CMC-Na, may be present in an amount of from about 0.1% to about 2%, e.g., about 0.7% or about 1.75% of the vehicle (by volume), e.g., in a concentration of about 1 mg/mL to about 30 mg/mL in the vehicle, e.g., about 7 mg/mL or about 17.5 mg/mL.

In a further aspect, the present invention provides a kit comprising microparticles of the invention and a vehicle of the invention. For example, the kit may comprise micro-particles comprising the exact amount of compound of the invention to be administered, e.g., as described below, and about 1 mL to about 5 mL, e.g., about 2 mL of the vehicle of the invention.

In one embodiment, the dry microparticles, optionally in admixture with an anti-agglomerating agent, may be filled into a container, e.g., a vial or a syringe, and sterilized e.g., using gamma-irradiation. Prior to (usually s.c. or i.m.) administration, the microparticles may be suspended in the container by adding a suitable vehicle, e.g., the vehicle described above. For example, the microparticles, optionally in admixture with an anti-agglomerating agent, a viscosity increasing agent and/or a tonicity agent, and the vehicle for suspension may be housed separately in a double chamber syringe. A mixture of the microparticles with an anti-agglomerating agent and/or a viscosity increasing agent and/or a tonicity agent, also forms part of the invention.

In another embodiment, under sterile conditions dry sterilized microparticles, optionally in admixture with an anti-agglomerating agent, may be suspended in a suitable vehicle, e.g., the vehicle described above, and filled into a container, e.g., a vial or a syringe. The solvent of the vehicle, e.g., the water, may then be removed, e.g., by freeze-drying or evaporation under vacuum, leading to a mixture of the microparticles and the solid components of the vehicle in the container. Prior to administration, the microparticles and solid components of the vehicle may be suspended in the container by adding a suitable vehicle, e.g., water, e.g., water for infusion, or preferably a low molarity phosphate buffer solution. For example, the mixture of the microparticles, optionally the anti-agglomerating agent, and solid components of the vehicle and the vehicle for suspension, e.g., water, may be housed separately in a double chamber syringe.

### III. Implants

It has been found that administration of implants comprising a poorly soluble salt of a bisphosphonate of the formula I embedded in a biocompatible pharmacologically acceptable polymer gives release of all or of substantially all of the active agent over an extended period of time, e.g., one week up to 18 months, especially for about 3 weeks to about 12 months, e.g. 3 months to about 12 months. The term “depot formulation” in the present disclosure therefore also refers to such implants.

The present invention in another aspect provides a process for the preparation of the implants of the invention comprising:
(i) preparation of a powder mixture of the poorly water soluble DS and the biodegradable polymer by cryo-milling with liquid nitrogen of both components together and/or using a organic solvent for a granulation step and removing this solvent again by a drying process;
(ii) filling a RAM extruder with the powder mixture (alternatively, a screw or a double-screw extruder is used);
(iii) heating the extruder walls to temperatures in the range of 50-120° C., in case of using poly(lactide-co-glycolide) as polymer matrix preferably 60-90° C.;
(iv) pushing the molten powder mixture through a pin hole of 1-4 mm diameter at small speed, preferably through a 1.5 mm pin hole with a speed of 5 mm/min.; and
(v) cutting the resulting sticks into shorter length depending on the anticipated dose, e.g., 20 mm.

For application the implants are placed in an applicator or trochar, sealed in aluminum foil and sterilized by using gamma-irradiation with a minimum dose of 25 kGy. These applicators are commercially available, e.g., by Rexam Pharma, Süddeutsche Feinmechanik GmbH(SFM) or Becton Dickerson.

### IV. Biocompatable Polymers

The polymer matrix of the depot formulations may be a synthetic or natural polymer. The polymer may be either a biodegradable or non-biodegradable or a combination of biodegradable and non-biodegradable polymers, preferably biodegradable.

By “polymer” is meant an homopolymer or a copolymer.

### Suitable Polymers Include:

(a) linear or branched polyesters which are linear chains radiating from a polyol moiety, e.g., glucose, e.g., a polyester, such as D-, L- or racemic polylactic acid, polyglycolic acid, polyhydroxybutyric acid, polycaprolactone, polyalkylene oxalate, polyalkylene glycol esters of an acid of the Kreb's cycle, e.g., citric acid cycle, and the like or a combination thereof,
(b) polymers or copolymers of organic ethers, anhydrides, amides and orthoesters, including such copolymers with other monomers, e.g., a polyanhydride, such as a copolymer of 1,3-bis-(p-carboxyphenoxy)-propane and a diacid, e.g., sebacic acid, or a copolymer of erucic acid dimer with sebacic acid; a polyorthoester resulting from reaction of an ortho-ester with a triol, e.g., 1,2,6-hexanetriol, or of a diketene acetal, e.g., 3,9-diethylidene-2,4,8,10-tetraoxaspiro[5,5]un-decane, with a diol, e.g., 1,6-dihexanediol, triethyleneglycol or 1,10-decanediol; or a polyester amide obtained with an amide-diol monomer, e.g., 1,2-di-(hydroxyacetamido)-ethane or 1,10-di-(hydroxyacetamido)decane; or
(c) polyvinylalcohol.

The polymers may be cross-linked or non-cross-linked, usually not more than 5%, typically less than 1%.

Preferred are polylactide-co-glycolide polymers (also named PLGA).

Table II lists examples of the polymers of the invention:

TABLE II

| Product Name | Polymer |
|---|---|
| D,L-POLYMI/D-GLUCOSE | Star-branched |
| | Poly(D,L-lactide-co-glycolide) |
| | 50:50/D-Glucose |
| Resomer ® R 202 H | Linear |
| | Poly(D,L-lactide) |
| | free carboxylic acid end group |
| Resomer ® R 202 | Linear |
| | Poly(D,L-lactide) |
| Resomer ® R 203 | Linear |
| | Poly(D,L-lactide) |
| Resomer ® RG 752 | Linear |
| | Poly(D,L-lactide-co-glycolide) |
| | 75:25 |
| Resomer ® RG 753 S | Linear |
| | Poly(D,L-lactide-co-glycolide) |
| | 75:25 |
| Lactel ® 100D020A | Linear |
| | Poly(D,L-lactide) |
| | free carboxylic acid end group |
| Lactel ® 100D040A | Linear |
| | Poly(D,L-lactide) |
| | free carboxylic acid end group |
| Lactel ® 100D040 | Linear |
| | Poly(D,L-lactide) |
| Lactel ® 100D065 | Linear |
| | Poly(D,L-lactide) |
| Lactel ® 85DG065 | Linear |
| | Poly(D,L-lactide-co-glycolide) |
| | 85:15 |
| Lactel ® 75DG065 | Linear |
| | Poly(D,L-lactide-co-glycolide) |
| | 75:25 |
| Lactel ® 65DG065 | Linear |
| | Poly(D,L-lactide-co-glycolide) |
| | 65:35 |
| Lactel ® 50DG065 | Linear |
| | Poly(D,L-lactide-co-glycolide) |
| | 50:50 |
| Lactel ® 50DG085 | Linear |
| | Poly(D,L-lactide-co-glycolide) |
| | 50:50 |
| Lactel ® 50DG105 | Linear |
| | Poly(D,L-lactide-co-glycolide) |
| | 50:50 |
| Medisorb ® 100 DL HIGH IV | Linear |
| | Poly(D,L-lactide) |
| Medisorb ® 100 DL LOW IV | Linear |
| | Poly(D,L-lactide) |
| Medisorb ® 8515 DL HIGH IV | Linear |
| | Poly(D,L-lactide-co-glycolide) |
| | 85:15 |
| Medisorb ® 8515 DL LOW IV | Linear |
| | Poly(D,L-lactide-co-glycolide) |
| | 85:15 |
| Medisorb ® 7525 DL HIGH IV | Linear |
| | Poly(D,L-lactide-co-glycolide) |
| | 75:25 |
| Medisorb ® 7525 DL LOW IV | Linear |
| | Poly(D,L-lactide-co-glycolide) |
| | 75:25 |
| Medisorb ® 6535 DL HIGH IV | Linear |
| | Poly(D,L-lactide-co-glycolide) |
| | 65:35 |
| Medisorb ® 6535 DL LOW IV | Linear |
| | Poly(D,L-lactide-co-glycolide) |
| | 65:35 |
| Medisorb ® 5050 DL HIGH IV | Linear |
| | Poly(D,L-lactide-co-glycolide) |
| | 50:50 |
| Medisorb ® 5050 DL LOW IV | Linear |
| | Poly(D,L-lactide-co-glycolide) |
| | 50:50 |

The preferred polymers of this invention are linear polyesters and branched chain polyesters. The linear polyesters may be prepared from alpha-hydroxy carboxylic acids, e.g., lactic acid and/or glycolic acid, by condensation of the lactone dimers. The preferred polyester chains in the linear or branched (star) polymers are copolymers of the alpha-carboxylic acid moieties, lactic acid and glycolic acid, or of the lactone dimmers, also referred to herein as PLGA. The molar ratio of lactide: glycolide of polylactide-co-glycolides in the linear or branched polyesters is preferably from about 100:0 to 40:60, more preferred from. 95:5 to 50:50, most preferred from 95:5 to 55:45.

Linear polyesters, e.g., linear polylactide-co-glycolides, preferably used according to the invention have a weight average molecular weight (Mw) between about 10,000 Da and about 500,000 Da, e.g., about 50,000 Da. Such polymers have a polydispersity M_{w}/Mₙ, e.g., between 1.2 and 2. Suitable examples include, e.g., poly(D,L-lactide-co-glycolide), linear poly (D,L-lactide) and liner-poly (D,L-lactide) free carboxylic acid end group, e.g., having a general formula —[(C₆H₈O₄)ₓ(C₄H₄O₄)_{y}]ₙ— (each of x, y and n having a value so that the total sum gives the above indicated Mws), e.g., those commercially-available, e.g., Resomers® from Boehringer Ingelheim, Lactel® from Durect, Purasorb® from Purac and Medisorb® from Lakeshore.

Branched polyesters, e.g., branched polylactide-co-glycolides, also used according to the invention may be prepared using polyhydroxy compounds, e.g., polyol, e.g., glucose or mannitol as the initiator. These esters of a polyol are known and described, e.g., in GB 2,145,422 B, the contents of which are incorporated herein by reference. The polyol contains at least 3 hydroxy groups and has a molecular weight of up to 20,000 Da, with at least 1, preferably at least 2, e.g., as a mean 3 of the hydroxy groups of the polyol being in the form of ester groups, which contain poly-lactide or co-poly-lactide chains. Typically 0.2% glucose is used to initiate polymerization. The branched polyesters (Glu-PLG) have a central glucose moiety having rays of linear polylactide chains, e.g., they have a star shaped structure.

The branched polyesters having a central glucose moiety having rays of linear polylactide-co-glycolide chains (Glu-PLG) may be prepared by reacting a polyol with a lactide and preferably also a glycolide at an elevated temperature in the presence of a catalyst, which makes a ring opening polymerization feasible.

The branched polyesters having a central glucose moiety having rays of linear polylactide-co-glycolide chains (Glu-PLG) preferably have an weight average molecular weight M_{w}, in the range of from about 10,000-200,000, preferably 25,000-100,000, especially 35,000-60,000, e.g., about 50,000 Da, and a polydispersity, e.g., of from 1.7-3.0, e.g., 2.0-2.5. The intrinsic viscosities of star polymers of M_{w} 35,000 or M_{w} 60,000 are 0.36 dL/g or 0.51 dL/g, respectively, in chloroform. A star polymer having a M_{w} 52,000 has a viscosity of 0.475 dl/g in chloroform.

The desired rate of degradation of polymers and the desired release profile for compounds of the invention may be varied depending on the kind of monomer, whether a homo- or a copolymer or whether a mixture of polymers is employed.

### V. Method of Treatment

The uses and methods of the present invention represent an improvement to existing therapy of various diseases, including diseases or disorders where abnormal (especially abnormally increased) bone turnover is found, also malignant diseases in which bisphosphonates are used to prevent or inhibit development of bone metastases or excessive bone resorption, and also especially for the therapy of inflammatory diseases such as rheumatoid arthritis and osteoarthritis. Use of bisphosphonates to embolise newly-formed blood vessels has been found to lead to suppression of tumors, e.g., solid tumors, and metastastes, e.g., bone metastases and even reduction in size of tumors, e.g., solid tumors, and metastases, e.g., bone metastases, after appropriate periods of treatment. It has been observed using angiography that newly-formed blood vessels disappear after bisphosphonate treatment, but that normal blood vessels remain intact. Further it has been observed that the embolised blood vessels are not restored following cessation of the bisphosphonate treatment. Also it has been observed that bone metastasis, rheumatoid arthritis and osteoarthritis patients experience decreased pain following bisphosphonate treatment.

Conditions of abnormal, e.g. abnormally increased, bone turnover which may be treated in accordance with the present invention include: treatment of (e.g. bone) cancer related abnormal bone turnover, treatment of postmenopausal osteoporosis, e.g., to reduce the risk of osteoporotic fractures; prevention of postmenopausal osteoporosis, e.g., prevention of postmenopausal bone loss; treatment or prevention of male osteoporosis; treatment or prevention of corticosteroid-induced osteoporosis and other forms of bone loss secondary to or due to medication, e.g., diphenylhydantoin, thyroid hormone replacement therapy; treatment or prevention of bone loss associated with immobilisation and space flight; treatment or prevention of bone loss associated with rheumatoid arthritis, osteogenesis imperfecta, hyperthyroidism, anorexia nervosa, organ transplantation, joint prosthesis loosening, and other medical conditions. For example, such other medical conditions may include treatment or prevention of periarticular bone erosions in rheumatoid arthritis; treatment of osteoarthritis, e.g., prevention/treatment of subchondral osteosclerosis, subchondral bone cysts, osteophyte formation, and of osteoarthritic pain, e.g., by reduction in intra-osseous pressure; treatment or prevention of hypercalcemia resulting from excessive bone resorption secondary to hyperparathyroidism, thyrotoxicosis, sarcoidosis, or hyper-vitaminosis D, dental resorptive lesions, pain associated with any of the above conditions, particularly, osteopenia, Paget's disease, osteoporosis, rheumatoid arthritis, osteoarthritis.

Especially useful (for human and veterinary use) is the treatment of one or more diseases (this term including conditions or disorders), involving abnormal bone turnover associated with diseases of bones and joints, for example
benign conditions such as osteoporosis, osteopenia, osteomyelitis, osteoarthritis, rheumatoid arthritis, bone marrow edema, bone pain, reflex sympathetic dystrophy, ankylosing spondylitis (aka Morbus Bechterev), Paget's disease of bone or periodontal disease,
malignant conditions such as hypercalcemia of malignancy, bone metastases associated with solid tumors and hematologic malignancies,
orthopedic conditions such as prosthesis loosening, prosthesis migration, implant fixation, implant coating, fracture healing, distraction osteogenesis, spinal fusion, avascular osteonecrosis, bone grafting, bone substitutes,
   or any combination of tow or more such conditions.

Appropriate dosage of the depot formulations of the invention will of course vary, e.g., depending on the condition to be treated (e.g., the disease type or the nature of resistance), the drug used, the effect desired and the mode of administration.

Specifically, with a depot formulation according to the invention satisfactory results are obtained on administration, e.g., parenteral administration, at dosages on the order of from about 0.2 mg to about 100 mg, e.g., 0.2 mg to about 35 mg, preferably from about 3 mg to about 100 mg of the compound of the formula I (calculated based on its free form) of the invention per injection per month or about 0.03 mg to about 1.2 mg, e.g., 0.03-0.3 mg per kg animal body weight per month. Suitable monthly dosages for patients are thus in the order of about 0.3 mg to about 100 mg of a compound of the formula I (calculated based on its free form, also here it is used in the form or the salt and/or crystal).

The pharmaceutical compositions in more general form which contain a compound of formula I as crystalline form of the free (e.g. zwitterionic) form or a poorly soluble salt of a compound of the formula I or especially a crystalline form of such a salt including a solvate, e.g. hydrate, especially a dihydrate, of such salt) as described hereinabove and -below are those for enteral such as oral, or rectal and parenteral, administration to warm-blooded animals, the pharmacological active ingredient being present alone or together with a pharmaceutically suitable carrier.

These further novel pharmaceutical compositions comprise e.g. from about 0.0001 to 80%, preferably from about 0.001 to 10%, of the active ingredient. Pharmaceutical compositions for enteral or parenteral administration are e.g. those in dosage unit forms such as dragees, tablets, capsules or suppositories, as well as ampoules, vials, pre-filled syringes. These pharmaceutical compositions are prepared in a manner known per se, for example by conventional mixing, granulating, confectioning, dissolving or lyophilising methods. For example, pharmaceutical compositions for oral administration can be obtained by combining the active ingredient with solid carriers, optionally granulating a resulting mixture and processing the mixture or granulate, if desired or necessary after the addition of suitable excipients, to tablets or dragee cores.

Suitable carriers are in particular fillers such as sugar, for example lactose, saccharose, mannitol or sorbitol, cellulose preparations and/or calcium phosphates, e.g. tricalcium phosphate or calcium biphosphate, and also binders such as starch pastes, e.g. maize, corn, rice or potato starch, gelatin, tragacanth, methyl cellulose and/or polyvinylpyrrolidone, and/or, if desired, disintegrators, such as the abovementioned starches, also carboxymethyl starch, crosslinked polyvinylpyrrolidone, agar, alginic acid or a salt thereof such as sodium alginate. Excipients are in particular glidants and lubricants, for example silica, talcum, stearic acid or salts thereof such as magnesium stearate or calcium stearate, and/or polyethylene glycol. Dragee cores are provided with suitable coatings which can be resistant to gastric juices, using inter alia concentrated sugar solutions which may contain gum arabic, talcum, polyvinylpyrrolidone, polyethylene glycol and/or titanium dioxide, shellac solutions in suitable organic solvents or mixtures of solvents or, for the preparation of coatings which are resistant to gastric juices, solutions of suitable cellulose preparations such as acetyl cellulose phthalate or hydroxypropyl methyl cellulose phthalate. Dyes or pigments can be added to the tablets or dragee coatings, for example to identify or indicate different doses of active ingredient.

Further pharmaceutical compositions for oral administration are dry-filled capsules made of gelatin or hypromellose and also soft sealed capsules consisting of gelatin and a plasticiser such as glycerol or sorbitol. The dry-filled capsules can contain the active ingredient in the form of granules, for example in admixture with fillers such as lactose, binders such as star-ches, and/or glidants such as talcum or magnesium stearate, and optionally stabilisers. In soft capsules, the active ingredient is preferably dissolved or suspended in a suitable liquid, such as a fatty oil, paraffin oil or a liquid polyethylene glycol, to which a stabiliser can also be added.

Suitable pharmaceutical compositions for rectal administration are e.g. suppositories, which consist of a combination of the active ingredient with a suppository base. Examples of suitable suppository bases are natural or synthetic triglycerides, paraffin hydrocarbons, polyethylene glycols and higher alkanols. It is also possible to use gelatin rectal capsules which contain a combination of the active ingredient with a base material. Suitable base materials are e.g. liquid triglycerides, polyethylene glycols and paraffin hydrocarbons.

Particularly suitable dosage forms for parenteral administration (which is especially preferred) are aqueous solutions of an active ingredient in water-soluble form, for example a water-soluble salt. The solution may be adjusted with inorganic or organic acids or bases to a physiologically acceptable pH value of about pH 4-9 or most preferably of about 5.5-7.5. The solutions further may be made isotonic with inorganic salts like sodium chloride, or organic compounds like sugars, sugar alcohols, or amino acids, most preferably with mannitol or glycerol. Suitable compositions are also suspensions of the active ingredient, such as corresponding oily injection suspensions, for which there are used suitable lipophilic solvents or vehicles such as fatty oils, for example sesame oil, or synthetic fatty acid esters, for example ethyl oleate or triglycerides, or aqueous injection suspensions which contain substances which increase the viscosity, for example sodium carboxymethyl cellulose, sorbitol and/or dextran, and optionally also stabilisers.

The present invention also relates to the forms of the compound of the formula I (including a salt, a crystalline form, and/or a depot formulation) preferably for the treatment of inflammatory conditions, primarily to diseases associated with impairment of calcium metabolism, e.g. rheumatic diseases and, in particular, osteoporosis.

Parenteral Doses below 0.1 μg/kg of body weight affect hard tissue metabolism only insignificantly. Long-term toxic side-effects may occur at doses of over 1000 μg/kg of body weight. The forms of compounds of formula I according to the invention can be administered orally, as well as subcutaneously, intramuscularly or intravenously in iso- or hypertonic solution. Preferred daily doses are, for oral administration, in the range from about 1 to 100 mg/kg, for intravenous, subcutaneous and intramuscular administration in the range from about 20 to 500 μg/kg.

The dosage of the forms of compounds of formula I (based on weight of the compound of formula I as such), however, variable and depends on the respective conditions such as the nature and severity of the illness, the duration of treatment and on the respective compound. Dosage unit form for parenteral, e.g. intravenous, administration contain e.g. from 10 to 300 μg/kg of body weight, preferably from 15 to 150 μg/kg body weight; and oral dosage unit forms contain e.g. from 0.1 to 5 mg, preferably from 0.15 to 3 mg per kg body weight. The preferred single dose for oral administration is from 10 to 200 mg and, for intravenous administration, from 1 to 10 mg. The higher doses for oral administration are necessary on account of the limited absorption. In prolonged treatment, the dosage can normally be reduced to a lower level after an initially higher dosage in order to maintain the desired effect. Parenteral, (e.g. intravenous or subcutaneous) doses may be administered intermittently at regular intervals between 1 and 52 times per year. Oral doses may be administered regularly on a daily, weekly, monthly or quarterly dosing regimen. For depot formulations according to the invention the dosages mentioned above are preferred.

The properties of the depot formulations, salts, crystal forms and pharmaceutical compositions of the invention may be tested in standard animal tests or clinical trials, e.g. as following:

The following publications (each of which is incorporated herein by reference, especially with regard to the description of the assays or methods mentioned below therein) describe various assays and methods that can be used to confirm the advantageous biological profile of the compounds of the formula I:

The effects of a single i.v. administration to mature, ovariectomized (OVX) rats as a model for postmenopausal osteoporosis in order to elucidate (1) the temporal changes in biochemical markers of bone turnover and femoral bone mineral density (BMD), (2) to measure changes of static and dynamic histomorphometric parameters, bone microarchitecture and mechanical strength, and (3) to assess the preventive effects of chronic treatment with a compound of the formula I on these parameters can be demonstrated as described in Calcif. Tissue Int. (2003) 72, 519-527. High activity can be found here.

The effect of a compound of the formula I (in the description of the following descriptions of possible biological assays this includes one or both of the salt forms as well as the crystal forms described herein) on synovial inflammation, structural joint damage, and bone metabolism in rats during the effector phase of collagen-induced arthritis (CIA) can be demonstrated as shown in ARTHRITIS & RHEUMATISM (2004), 50(7), 2338-2346.

The effect of a compound of the formula I on bone ingrowth can be examined in an animal model in which porous tantalum implants are placed bilaterally within the ulnae of dogs as described in J. Bone Joint Surg. (2005), 87-B, 416-420.

Inhibition of skeletal tumor growth in a mouse model can be demonstrated in accordance with the method described in J. Natl. Cancer. Inst. (2007), 99, 322-30.

The x-ray structure of compounds of the formula I when bound to farnesyl pyrophosphate synthase can be obtained by or in analogy to the methods described in Chem. Med. Chem. (2006), 1, 267-273. Human FPPS, a homodimeric enzyme of 41-kDa subunits, catalyzes the two-step synthesis of the C15 metabolite farnesyl pyrophosphate (FPP) from the C5 isoprenoids dimethylallyl pyrophosphate (DMAPP) and isopentenyl pyrophosphate. FPP is required for the posttranslational prenylation of essential GTPase signalling proteins such as Ras and Rho and is also a precursor for the synthesis of cholesterol, dolichol, and ubiquinone.

For example, in a cell-free in vitro assay the superiority of compounds of the formula I over compounds already known can be shown. Briefly, the reaction proceeds in the presence of enzyme and an inhibitor of the formula I, and the reaction product (farneysyl pyrophosphate) is quantified by LC/MS/MS.

In detail, the inhibitor and enzyme are pre-incubated before adding the substrates

The assay is a label-free assay for farnesyl pyrophosphate synthase (FPPS) based on LC/MS/MS. This method quantifies in-vitro untagged farnesyl pyrophosphate (FPP) and is suitable for high throughput screening (HTS) to find inhibitors of FPPS and for the determinations of IC50 values of candidate compounds. The analysis time is 2.0 minutes with a total cycle time of 2.5 minutes. The analysis can be formatted for 384-well plates resulting in an analysis time of 16 hours per plate.

### Reagents:

Pentanol, methanol, and isopropyl alcohol are HPLC grade and obtained from Fisher Scientific. DMIPA is from Sigma-Aldrich. Water is from an in-house Milli-Q system. The assay buffer (20 mM HEPES, 5 mM MgCl₂ and 1 mM CaCl₂) is prepared by dilution from 1 mM stock solutions obtained from Sigma-Aldrich. Standards of geranyl pyrophosphate (GPP), isoprenyl pyrophosphate (FPP), and farnesyl S-thiolopyrophosphate (FSPP) are from Echelon Biosciences (Salt Lake City, Utah). Human farnesyl pyrophosphate synthase (FPPS, Swissprot ID: P14324) (13.8 mg/mL) is prepared as described by Rondeau et al (ChemMedChem 2006, 1, 267-273.

### Assay:

LC/MS/MS analyses are performed on a Micromass Quattro Micro tandem quadrupole mass analyser (Waters Corp., Milford, Mass., USA) interfaced to an Agilent 1100 binary LC pump Agilent Technologies, Inc., Santa Clara, Calif., USA). Injection is performed with a CTC Analytics autosampler (Leap Technologies Inc., Carrboro, N.C., USA) using an injection loop size of 2.5 μL. Chromatography is performed on a Waters 2.1×20 mm Xterra MS C18 5 μm guard column (P/N186000652) (Waters Corp., Milford, Mass., USA) contained in a guard column holder (P/N 186000262) using 0.1% DMIPA/methanol as solvent A and 0.1% DMIPA/water as solvent B (DMIPA is dimethylisopropylamine). The gradient is 5% A from 0.00 to 0.30 min., 50% A at 0.31 min., 80% A at 1.00 min., and 5% A from 1.01 to 2.00 min. The flow rate is 0.3 mL/min, and the flow is diverted to waste from 0.00 to 0.50 min and again from 1.20 to 2.00 min.

The Multiple Reaction Monitoring (MRM) transitions monitored are 381−>79− for FPP and 397−>159− for FSPP at a collision energy of 22 eV and a collision cell pressure of 2.1×10⁻³ mbar of Ar. The dwell time per transition is 400 msec with a span of 0.4 Da. The inter-channel delay and interscan delay are both 0.02 sec. Other mass spectrometric operating parameters are: capillary, 2.0 kV; cone, 35 V; extractor, 2.0 V, source temp., 100° C.; desolvation gas temp., 250° C.; desolvation gas flow, 650 L/hr; cone gas flow, 25 L/hr; multiplier, 650 V.

The total cycle time per sample is 2.5 minutes. Since the analysis is formatted for 384-well plates, a plate is analyzed in 16 hours. The chromatograms are processed using Quanlynx software, which divides the area of individual FPP peaks by the area of the FSPP peaks (internal standard). The resulting values are reported as the relative response for the corresponding sample well.

### FPPS Assay Procedure

Into each well of a 384-well plate, 5 μL of compound in 20% DMSO/water is placed. 10 μL of FPPS (diluted 1 to 80000 with assay buffer) is added to each well and allowed to pre-incubate with the compound for 5 minutes. At that time, 25 μL of GPP/IPP (5 μM each in assay buffer) is then added to start the reaction. After 30 minutes the reaction is stopped by addition of 10 μL of 2 μM FSPP in 2% DMIPA/IPA. The reaction mixture is then extracted with 50 μL of n-pentanol using vortex mixing. After phase separation, 25 μL of the upper (n-pentanol) layer is transferred to a new 384-well plate and the pentanol is evaporated using a vacuum centrifuge. The dried residue is reconstituted in 50 μL of 0.1% DMIPA/water for analysis by the LC/MS/MS method.

FSPP is used as the internal standard for the mass spectra. A phosphate moiety generates an (M-H)-ion as the base peak in the spectra.

The compounds of the invention preferably, in this test system, have an IC₅₀ in the range from 0.8 to 10 nM, the preferred ones preferably from 0.9 to 3.3 nM, (e.g. in the case of experiments with [2-(5-ethyl-imidazol-1-yl)-1-hydroxy-1-phosphono-ethyl]-phosphonic acid in the range from 2.4 to 3.1 nM). Especially, they, e.g. [2-(5-ethyl-imidazol-1-yl)-1-hydroxy-1-phosphono-ethyl]-phosphonic acid, show a surprising superiority over compounds in the prior art. The utility of the assay for IC₅₀ determinations is validated using zoledronic acid, a known bisphosphonate inhibitor of FPPS.

The depot formulation, the salts and the crystal forms, as well as the compositions of the invention, are well-tolerated.

The invention also relates to the embodiments given in the claims, especially the dependent claims, so that said claims are incorporated here by reference, as well as especially to the embodiments of the invention provided in the following Examples.

Also the Abstract is incorporated here by reference, also disclosing embodiments of the invention.

### General Preparation of Compounds of the Formula I:

A compound of the formula I can be prepared according to methods that, for different compounds, are known in the art. For example, based at least on the novel products obtained and/or the novel educts employed, a novel process is preferred comprising reacting a carboxylic acid compound of the formula II,

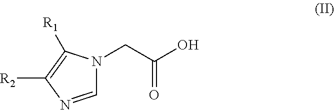

wherein R₁ and R₂ are as defined for a compound of the formula I, with phosphorous oxyhalogenide to give a compound of the formula I, or a salt thereof,

and, if desired, converting an obtainable free compound of the formula I into its salt, converting an obtainable salt of a compound of the formula I into the free compound and/or converting an obtainable salt of a compound of the formula I into a different salt thereof.

As phosphorous oxyhalogenide, phosphorous oxychloride (POCl₃) is especially preferred. The reaction preferably takes place in a customary solvent or solvent mixture, e.g. in an aromatic hydrocarbon, such as toluene, at preferably elevated temperatures, e.g. in the range from 50° C. to the reflux temperature of the reaction mixture, e.g. from (about) 80 to (about) 120° C.

The starting materials of the formula II can, for example preferably, be obtained by saponifying a compound of the formula III,

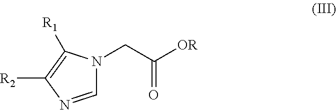

wherein R₁ and R₂ are as defined for a compound of the formula I and R is unsubstituted or substituted alkyl, especially lower alkyl or phenyl-lower alkyl, in the presence of an appropriate acid, e.g. a hydrohalic acid, such as hydrochloric acid, preferably in the presence of an aqueous solvent, such as water, at preferably elevated temperatures, e.g. in the range from (about) 50 to (about) 100° C., e.g. from 80 to 100° C., to give the compound of the formula II, or a salt thereof.

A compound of the formula III can, for example preferably, be obtained by reacting an imidazole compound of the formula IV,

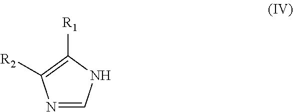

wherein R₁ and R₂ are as defined for a compound of the formula I, with an ester of the formula V,

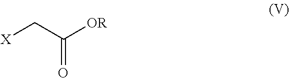

wherein R is as defined for a compound of the formula III and X is halogen, especially fluoro, chloro, iodo or especially bromo, lower-alkanesulfonyloxy or toluenesulfonyloxy, preferably in the presence of a strong base, such as an alkaline metal alcoholate, especially potassium tert-butylate, in an appropriate solvent or solvent mixture, e.g. a cyclic ether, such as tetrahydrofurane, preferably at temperatures in the range from (about)-10 to (about) 80° C., e.g. from 20 to 30° C. Where required, resulting mixtures of compounds of the formula III (wherein in one compound R₁ is C₂-C₅-alkyl and R₂ is hydrogen, in the other R₂ is C₂-C₅-alkyl and R₁ is hydrogen) can be separated e.g. by chromatographic methods, differential crystallisation or the like.

Starting materials of the formulae IV and V, as well as any other starting materials employed not described so far, can be obtained by methods that are known in the art or in analogy thereto, are commercially available and/or can be made in analogy to methods described herein.

The following Examples serve to illustrate the invention without limiting its scope.

The following compounds of the formula I are obtained as described in the following Reference Examples:

If not mentioned otherwise, temperatures are given in degree Celsius (° C.). Where no temperature is mentioned, the reaction or other method step takes place at room temperature.

### Abbreviations

Ac. acetyl
aq. Aqueous
DMSO dimethyl sulfoxide
Et ethyl
h hour(s)
HPLC high performance liquid chromatography
KOtBu potassium tert-butylate
Me methyl
ml milliliter(s)
NMR Nuclear Magnetic Resonance
rt room temperature
THF tetrahydrofurane
   4- and 5-Ethylimidazole and all other imidazole derivatives are prepared according to D. Horne et al., Heterocycles, 1994, Vol. 39, No. 1, p. 139-153.

### Reference-Example 1

### [2-(4-Ethyl-imidazol-1-yl)-1-hydroxy-1-phosphono-ethyl]-phosphonic acid (Also Named Compound A or Cpd. A Hereinafter)

650 mg (3.38 mmol) (4-ethyl-imidazol-1-yl)-acetic acid are dissolved in 15 ml toluene at it under nitrogen. 852 mg (3 mmol) H₃PO₃ are added and the mixture is heated to 80° C. 0.936 ml (3 mmol) POCl₃ are added drop wise. The resulting mixture is heated to 120° C. and stirred overnight. The solvent is decanted off, 15 ml 6N HCl is added and the mixture is heated for three hours at reflux.

The resulting pale yellow solution is concentrated in vacuo. After dilution with acetone (25 ml) the mixture is stirred vigorously with acetone (5×25 ml) until a grey solid is formed. The grey solid is dried in high vacuo and crystallized from EtOH/water to give the title compound. HPLC-MS: t=0.31 min, (M-H)−=299; ¹H-NMR (D₂O/NaOD): δ=1.07 (t, 3H), 2.53 (q, 2H), 4.45 (t, 2H), 7.08 (s, 1H), 8.40 (s, 1H), ³¹P-NMR (D₂O/NaOD): δ=15.04 ppm

### Synthesis Overview:

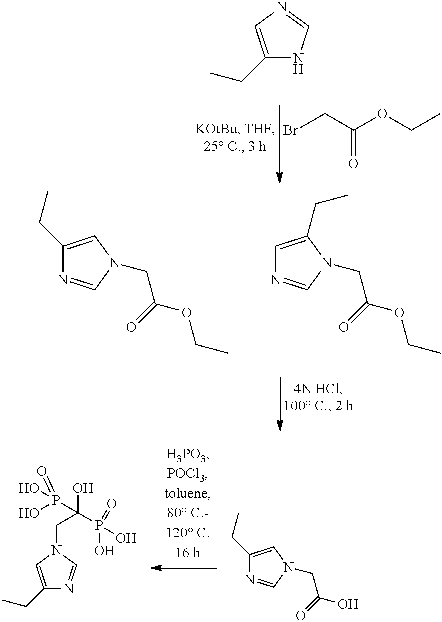

### HPLC-MS Conditions:

| Column: | XTerra (Waters Corp., Milford, MA, USA) 3 × 30 mm, | |
|---|---|---|
| | 2.5 μm, C18 | |
| Solvent A: | water, 5% acetonitrile, 1% HCOOH | |
| Solvent B: | acetonitrile, 1% HCOOH | |
| | min | % B |
| Gradient: | 0.0 | 01 |
| | 0.5 | 01 |
| | 2.5 | 30 |
| | 3.5 | 95 |
| | 4.5 | 95 |
| | 4.9 | 01 |

The starting materials are prepared as follows:

### Step 1: (4-Ethyl-imidazol-1-yl)-acetic acid ethyl ester and (5-ethyl-imidazol-1-yl)-acetic acid ethyl ester

5.02 g (50 mmol) of 4-ethylimidazole are dissolved in 100 ml THF at rt under nitrogen. 5.9 g (52 mmol) KOtBu is added and the reaction is stirred for 2 h at rt. 6.3 ml (55 mmol) ethyl bromoacetate is added drop wise over a period of 30 min and the resulting mixture is stirred at it for 2.5 h. 20 ml H₂O and 130 ml AcOEt are added, the organic layer is separated and the aq. layer is washed again 2× with 100 ml AcOEt. The combined organic layer is washed with brine, dried over MgSO₄ and concentrated in vacuo. The reaction is purified by Flash-chromatography (silica gel, MeOH/methylen chloride) to give (4-ethyl-imidazol-1-yl)-acetic acid ethyl ester and (5-ethyl-imidazol-1-yl)-acetic acid ethyl ester, respectively.

(4-Ethyl-imidazol-1-yl)-acetic acid ethyl ester: HPLC-MS: t=0.60 min; 100 area %, MH+=183; ¹H-NMR (d₆-DMSO) δ=1.09 (t, 3H), 1.18 (t, 3H), 2.43 (q, 2H), 4.13 (q, 2H), 4.83 (s, 2 H), 6.78 (s, 1H), 7.43 (s, 1H)

(5-Ethyl-imidazol-1-yl)-acetic acid ethyl ester: HPLC-MS: t=0.72 min, 100 area %, MH+=183; ¹H-NMR (d₆-DMSO): δ=1.12 (t, 3H), 1.18 (t, 3H), 2.40 (q, 2H), 4.14 (q, 2H), 4.85 (s, 2H), 6.61 (s, 1H), 7.48 (s, 1H)

### Step 2: (4-Ethyl-imidazol-1-yl)-acetic acid

1.7 g (9.5 mmol) of (4-ethyl-imidazol-1-yl)-acetic acid ethyl ester are dissolved in 47 ml (190 mmol) 4N HCl and the mixture is heated to reflux. After 2h the mixture is cooled to it and the solvent is removed in vacuo. The resulting product is used without further purification. MS: MH+=155, ¹H-NMR (DMSO): δ=1.18 (t, 3H), 2.65 (q, 2H), 5.07 (s, 2H), 7.43 (d, 1H). 9.0 (d, 1H)

### Reference-Example 2

### [2-(5-Ethyl-imidazol-1-yl)-1-hydroxy-1-phosphono-ethyl]-phosphonic acid

[2-(5-Ethyl-imidazol-1-yl)-1-hydroxy-1-phosphono-ethyl]-phosphonic acid is synthesized according to the synthesis outlined above from the corresponding (5-ethyl-imidazol-1-yl)-acetic acid ethyl ester which is the second product of step 1 in Example 1.

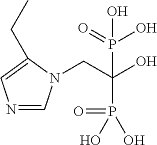

HPLC-MS: t=0.32 min, (M-H)−=299; ¹H-NMR (D₂O/NaOD): δ=1.10 (t, 3H), 2.63 (q, 2H), 4.43 (t, 2H), 6.95 (s, 1H), 8.54 (s, 1H), ³¹P-NMR (D₂O/NaOD): δ=14.96 ppm

In analogy to the above described procedures the following compounds are prepared:

### Reference-Example 3

### [2-(4-Propyl-imidazol-1-yl)-1-hydroxy-1-phosphono-ethyl]-phosphonic acid

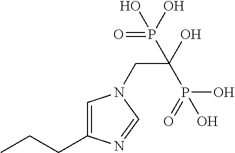

HPLC-MS: t=0.44 min, (M-H)−=313.1; ¹H-NMR (D₂O/NaOD): δ=0.78 (t, 3H), 1.52 (m, 2H), 2.52 (t, 2H), 4.50 (t, 2H) 7.13 (s, 1H), 8.45 (s, 1H); ³¹P-NMR (D₂O/NaOD) δ=15.25 ppm

### Reference-Example 4

### [2-(5-Propyl-imidazol-1-yl)-1-hydroxy-1-phosphono-ethyl]-phosphonic acid

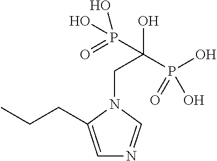

HPLC-MS: t=0.46 min, (M-H)−=313.1; ¹H-NMR (D₂O/NaOD): δ=0.81 (t, 3H), 1.51 (m, 2H), 2.60 (t, 2H), 4.44 (t, 2H), 6.96 (s, 1H), 8.54 (s, 1H); ³¹P-NMR (D₂O/NaOD) δ=15.06 ppm

### Reference-Example 5

### [2-(4-Butyl-imidazol-1-yl)-1-hydroxy-1-phosphono-ethyl]-phosphonic acid

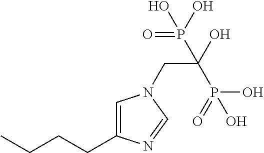

HPLC-MS: t=0.56 min, (M-H)−=327.2; ¹H-NMR (D₂O/NaOD): δ 0.73 (t, 3H), 1.17 (m, 2H), 1.46 (m, 2H), 2.51 (t, 2H), 4.44 (t, 2H) 7.09 (s, 1H), 8.40 (s, 1H); ³¹P-NMR (D₂O/NaOD): δ=14.98 ppm

### Reference-Example 6

### [2-(5-Butyl-imidazol-1-yl)-1-hydroxy-1-phosphono-ethyl]-phosphonic acid

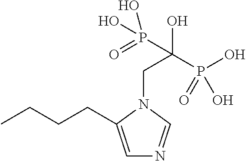

HPLC-MS: t=0.44 min, (M-H)−=327.2; ¹H-NMR (D₂O/NaOD): δ=0.79 (t, 3H), 1.27 (m, 2H), 1.51 (m, 2H), 2.67 (t, 2H), 4.49 (t, 2H), 6.99 (s, 1H), 8.58 (s, 1H); ³¹P-NMR (D₂O/NaOD): δ=15.16 ppm

### Reference-Example 7

### [1-Hydroxy-2-(4-isopropyl-imidazol-1-yl)-1-phosphono-ethyl]-phosphonic acid

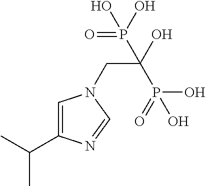

HPLC-MS: t=0.42 min, (M-H)−=313; ¹H-NMR (d₆-DMSO): δ=1.13, 1.15 (d, 6H), 2.86-2.95 (m, 1H), 4.49 (t, 2H), 7.12 (s, 1H), 8.46 (s, 1H); ³¹P-NMR (d₆-DMSO): δ=15.35 ppm

### Reference-Example 8

### [[1-Hydroxy-2-(5-isopropyl-imidazol-1-yl)-1-phosphono-ethyl]-phosphonic acid

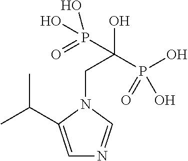

HPLC-MS: t=0.40 min, (M-H)−=313; ¹H-NMR (d₆-DMSO): δ=1.10, 1.12 (d, 6H), 3.12-3.19 (m, 1H), 4.52 (t, 2H), 7.01 (s, 1H), 8.56 (s, 1H); ³¹P-NMR (d₆-DMSO): δ=15.24 ppm

### Reference-Example 9

### [{2-[4-(1-Ethyl-propyl)-imidazol-1-v-1′-1-hydroxy-1-phosphono-ethyl}-phosphonic acid

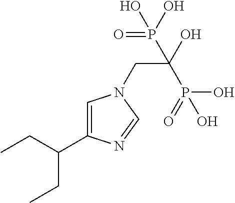

HPLC-MS: t=0.55 min, (M-H)−=341; ¹H-NMR (d₆-DMSO): d=0.80 (m, 6H), 1.50-1.75 (m, 4H), 2.49-2.60 (m, 1H), 4.52 (bs, 2H), 7.40 (s, 1H), 8.90 (s, 1H).

### Reference-Example 10

### {2-[5-(1-Ethyl-propyl)-imidazol-1-yl]-1-hydroxy-1-phosphono-ethyl}-phosphonic acid

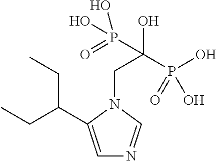

¹H-NMR (d₆-DMSO): d=0.77 (m, 6H), 1.40-1.60 (m, 4H), 2.97 (t, 1H), (4.44 (t, 2H), 6.60 (s, 1H), 7.92 (s, 1H).

### Example 1

### Manufacturing process for Ca-[2-(5-Ethyl-imidazol-1-yl)-1-hydroxy-1-phosphono-ethyl]-phosphonic acid salt (1:2)

3.5 g (11.67 mmol) of [2-(5-ethyl-imidazol-1-yl)-1-hydroxy-1-phosphono-ethyl]-phosphonic acid are dissolved in 540 ml of deionized water at 90° C. To this solution, within 1 minute a 90° C. hot solution of 667 mg (5.83 mmol) calcium chloride in 10 ml of water is added. The reaction mixture is cooled to 20° C. over 14 h and the suspension is filtered. The solid is washed with 2×50 ml of ice water and dried at 60° C. and 5 mbar. The calcium-[2-(5-ethyl-imidazol-1-yl)-1-hydroxy-1-phosphono-ethyl]-phosphonic acid salt (1:2) is obtained.

### Example 2

### Manufacturing process for Zn-[2-(5-Ethyl-imidazol-1-yl)-1-hydroxy-1-phosphono-ethyl]-phosphonic acid salt (1:2)

3.5 g (11.67 mmol) of [2-(5-ethyl-imidazol-1-yl)-1-hydroxy-1-phosphono-ethyl]-phosphonic acid are dissolved in 540 ml deionized water at 90° C. To this solution, within 1 minute a 90° C. hot solution of 811 mg (5.83 mmol) zinc chloride is 10 ml of water is added. The reaction mixture is cooled to 20° C. and dried at 60° C. to yield the zinc-[2-(5-ethyl-imidazol-1-yl)-1-hydroxy-1-phosphono-ethyl]-phosphonic acid salt (1:2).

### Example 3

### Micronisation of the Salts from Examples 1 and 2 and Manufacturing of Microparticles According to the Invention with the Micronized Salts

### a) Micronisation

### (i) Milling

The dry Calcium-salt of Example 1 and the dry Zinc-salt of Example 2 are milled on an ceramic air-jetmill (5 bar milling gas pressure).

### (ii) Resulting Particles

Before milling of the Calcium salt, the particles have a size of up to about 150 μm. After the milling, the particles have a size smaller than 10 μm according to microscopy.

In the case of the Zinc salt, the particles before milling have a size of up to about 100 μm. After the milling, the particles display sizes below 5 μm.

b) Manufacturing of Microparticles 3.1 g of a PLGA 50:50 with an inherent viscosity of 0.38 dL/g (Lactel®) are dissolved in 15.5 mL dichloromethane to form a clear 20% (m/V) PLGA solution. 0.9 g Ca-[2-(5-ethyl-imidazol-1-yl)-1-hydroxy-1-phosphono-ethyl]-phosphonic acid (1:2) salt from Example 1 (assay of free acid 89.0%) are dispersed into the PLGA solution using a high-shear force mixer (Ultra Turrax, S25N-10G) at 20′000 rpm for 4 min under cooling in an ice-water bath. The resulting suspension is referred to as the organic phase.

25 g of a polyvinyl alcohol 18-88 (PVA hereinafter), 11.3 g of sodium acetate trihydrate and 25.0 g glacial acetic acid are dissolved in 5 L water. This 0.5% PVA-100 mM Acetate buffer solution with a pH of 4 is referred to as aqueous phase.

The organic phase is mixed with the aqueous phase through an in-line high shear force device with two inflows and one outflow at a flow rate ratio of 30:600 mL/min and at 3800 rpm. The resulting emulsion is collected in a double walled reactor bearing already a starting volume of 170 mL aqueous phase under stirring with a propeller stirrer at 400 rpm.

The dichloromethane is removed by evaporation which is facilitated by continuous stirring of the batch with 400 rpm, slowly heating up the batch to 50° C. within 5 hours, keeping this temperature for further 2 hours. During all this time the gas phase near the surface of the emulsion is exchanged using vacuum.

After cooling down to room temperature again the formed microparticles sediment for 12 hours. The supernatant is removed to large extend. The microparticles are resuspended again in the remaining supernatant and isolated by filtration over 5 μm. The microparticles are washed 4 times with ca. 50 mL water and dried in vacuum for 3 days. Finally, the dried microparticles are desagglomerated by sieving through 140 μm mesh size. 2.32 g microparticles are obtained as fine white powder. The electron microscopic image showed perfectly spherical particles of smooth surface. The microparticle size distribution was found by laser light diffraction as follows: ×10: 15.6 μm, ×50: 35.8 μm, ×90: 53.7 μm. A drug substance assay of 14.7% is found by HPLC which corresponds to an encapsulation efficacy of 74%. After 24 h only 1.9% of the drug substance is released in an in vitro release test in a buffer of pH 7.4 at 37° C., which shows that this formulation advantageously avoids a too early release.

The following table summarize the key formulation, process and analytical data of example 1 and for further examples 2-4 of [2-(5-ethyl-imidazol-1-yl)-1-hydroxy-1-phosphono-ethyl]-phosphonic acid salt (composition see table) microparticles prepared analogously to example 1.

TABLE 1
Formulation, process and analytical data

| | [2-(5-ethyl- | Assay of | PLGA L:G ratio | | | | | | | | | | | |
|---|---|---|---|---|---|---|---|---|---|---|---|---|---|---|
| | imidazol-1- yl)-1- | free acid | Inhe-rent | | | | | Drug load | | | | | | |
| | hydroxy-1-phos- | Amount | vis-co-sity | | | | | (theoretical | | | | | | |
| | phono-ethyl]- | of DS | Amount of | | Particle size | | | encap-su- | Drug release (cumulative | | | | | |
| Example | phosphonic | used for | PLGA used for | Yield | distribution (um) | | | lation effi- | release data after x days) | | | | | |
| 3 | acid salt | 4 g batch | 4 g batch | (g) | X10 | X50 | X90 | ca-cy) | 1 | 2 | 3 | 7 | 14 | 21 |
| A | Ca (1:2) | 89% | 50:50 | 2.32 | 15.6 | 35.9 | 53.7 | 14.73% | 1.9 | 4.6 | 8.2 | 24.6 | 52.6 | 59.7 |

| | | | | |
|---|---|---|---|---|
| 0.9 | g | 0.38 | dL/g | (74%) |

| | |
|---|---|
| 3.1 | g |

| | | | | | | | | | | | | | | |
|---|---|---|---|---|---|---|---|---|---|---|---|---|---|---|
| B | Ca (1:2) | 89% | 75:25 | 2.32 | 14.9 | 35.8 | 55.2 | 4.85% | 19.7 | 19.8 | 20.3 | 20.4 | 26.3 | 31.7 |

| | | | | |
|---|---|---|---|---|
| 0.9 | g | 0.4 | dL/g | (23%) |

| | |
|---|---|
| 3.1 | g |

| | | | | | | | | | | | | | | |
|---|---|---|---|---|---|---|---|---|---|---|---|---|---|---|
| C | Zn (1:2) | 85.6% | 50:50 | 3.21 | 8.7 | 33.2 | 51.2 | 13.92% | 47.5 | 46.0 | 46.9 | 47.1 | 50.8 | 53.4 |

| | | | | |
|---|---|---|---|---|
| 0.93 | g | 0.38 | dL/g | (70%) |

| | |
|---|---|
| 3.07 | g |

| | | | | | | | | | | | | | | |
|---|---|---|---|---|---|---|---|---|---|---|---|---|---|---|
| D | Zn (1:2) | 85.6% | 75:25 | 2.82 | 17.1 | 37.5 | 57.0 | 14.19% | 51.5 | 50.1 | 51.4 | 50.1 | 52.4 | 56.4 |

| | | | | |
|---|---|---|---|---|
| 0.93 | g | 0.4 | dL/g | (71%) |

| | |
|---|---|
| 3.07 | g |

DS stands for drugs substance, L:G for the molar ratio of lactic acid to glycolide in the co-monomer. In examples 1 and 3 Lactel® is used as PLGA, in examples 2 and 4 Resomer® RG 753 S (see Table II above).

This shows quite clearly that in this case the Ca-salt microparticle formulation of Example 3A shows the most advantageous release kinetics with only very low release on day 1 and less than 60% release until day 21 of the compound of the formula I.

Using scanning electron microscopic images (the samples are sputtered with gold-palladium and investigated by a scanning electron microscopy of the microparticles of the formulations of Example 3A, 3B, 3C and 3D), it becomes evident that in the case of the Zn-salt particles (Examples 3C and 3D) the drug substance is apparently not encapsulated at all or only to a minor degree: Drug substance particles can be observed on the surface of the particles, and no drug substance can be seen in the polymer matrix in cross sections. For the surface representation, the microparticles are transferred to suitable object holders, sputtered with 20 nm gold and examines with the scanning electron microscope (Camscan CS 24/EO, Id. G.16.MIK.S008). For the cross section views, cross sections are prepared by embedding the particles in Araldite F (trademark from Ciba Specialty Chemicals, Basle, Switzerland; epoxy resin) and cutting semi-thin sections (thickness approximately 1 μm). The sections are sputtered with gold and also examined in the SEM.

In contrast, the images of the Ca salt microparticles (Examples 3A, 3B) demonstrate the more efficous encapsulation compared to the Zn salt. However, still some drug substance particles can be seen at the surface of the PLGA 75:25 formulation (Example 3B) which explains the high burst effect (high first 24h release). In contrast, the Ca salt formulation with PLGA 50:50 shows no drug substance at all at the surface. In the cross-section of particles the drug substance particles can be observed.

### Example 4

### Tolerability Study of Calcium-salt of Cpd. A Microparticles in Rats after s.c. Administration

The microparticles of Example 3A are suspended in a vehicle containing sodium carboxy methylcellulose, D-mannitol, Pluronics F68® (poloxamer 188, a copolymer of ethyleneoxide and propylene oxide, BASF AG, Ludwigshafen, Germany) and water for injection. 200 microliters of these suspensions were injected subcutaneously to the shaved skin at the left dorsal side of 8 weeks old female virgin Wistar rats (body weight approximately 220 g). In this way 1 mg dose (per animal) of the calcium—Cpd.A microparticles (Example 3A) and 2 mg dose (per animal) are applied to a group of 6 animals. The thickness of the skin will be measured by a micro-caliper at the side of injection and the contra-lateral non-injected side. As reference a suspension of non-encapsulated drug substance is injected in a dose of 60 microgram. In addition, placebo microparticles made out of PLGA 50:50 are also injected as control.

It can be shown whether no irritation is caused by the microparticle formulation according to the invention.

### Example 5

### The Production of Implants with Cpd. A-Calcium salt

1.5 g of the micronized calcium salt of Cpd. A (1:2 salt) and 10.7 g of a PLGA 50:50 (IV 0.65 dL/g) are mixed thoroughly through cryo-milling in liquid nitrogen. The resulting fine powder is extruded at 90° C. through a ram extruder with a speed of 5 mm/min. The implants of 1.5 mm diameter are cut at 2 cm length. The implants are placed in applicators, sealed in aluminum foil and finally sterilized by using gamma-irradiation with a dose of 30 kGy.

In the following Examples, X-ray powder diffraction patterns are measured on a Bruker D8 Advanced Series 2 Diffractometer, Detector: PSD Vantec-1 with Cu Ka (1.54 A) radiation. Parameters are as described in the Examples, respectively.

### Example 6

### Crystals of Cpd. a in Free Form (Internal Zwitterionic Salt Form)

The X-ray diffractogram of the zwitterionic salt of Cpd. A (which is obtainable as in Reference-Example 1) is obtained (elemental analysis C 27.9% (28.0), H 4.6% (4.7), N 9.4% (9.3%), P 20.5% (20.6%) (theoretical values in brackets) and yields the following Peaks (see also FIG. 1):

TABLE a
Powder X-Ray Diffraction Peaks for the crystalline form
of the zwitterions of Cpd. A from Reference-Example 1:

| ° deg 2⊖ | d-spacing (Å) | Relative intensity (%) |
|---|---|---|
| 10.5 | 8.39 | Medium |
| 13.1 | 6.74 | Medium |
| 14.7 | 6.03 | Medium |
| 17.2 | 5.14 | Medium |
| 23.5 | 3.78 | High |
| 25.2 | 3.54 | High |
| 34.4 | 2.60 | Low |

Type: 2Th locked—Start: 2.000°-End: 40.030°—Step: 0.017°-Step time: 107.s—Temp.: 25° C. (Room)-Time Started: 0 s-2-Theta: 2.000°.
The melting point (m.p.) of the zwitterionic form is 237° C.

### Example 7

### Crystals of Cpd. a in Ca-Salt (1:2) Form

a) Process for making the Crystalline Form of the 1:2 Calcium Salt of Cpd. A (1 equivalent Ca: 2 equivalents Cpd. A):

In a 1000 mL 3-necked flask with mechanical stirrer, 3.5 g of Cpd. A and 540 mL water are added. This mixture is heated to about 90° C. until everything goes into solution. To this clear solution a solution of 667 mg calcium chloride dihydrate in 10 mL water is added. A white precipitate appears and the mixture is then cooled to room temperature and stirred over night. The precipitated Ca salt is then isolated by filtration, washed with cold water and dried in a vacuum oven at 60° C. over night. This the Ca salt of a composition which corresponds to the dihydrate, m.p. >230° C. The Ca-content of the salt is 5,8% (theory 5.9%).

Here the calcium salt of Cpd. A has a stoichiometry of one calcium and two Cpd. A molecules.

b) The X-ray diffractogram of Ca-salt of Cpd. A as obtainable under a) yields the following Peaks (see also FIG. 2):

TABLE b
Powder X-Ray Diffraction Peaks for the Crystalline
Form of the 1:2 Calcium Salt of Cpd. A

| ° deg 2⊖ | d-spacing (Å) | Relative intensity (%) |
|---|---|---|
| 7.9 | 11.25 | high |
| 10.6 | 8.33 | low |
| 12.1 | 7.31 | medium |
| 25.7 | 3.46 | medium |
| 27.4 | 3.25 | medium |
| 29.2 | 3.05 | low |

Type: 2Th locked—Start: 2.000°-End: 40.030°—Step: 0.017°-Step time: 107.s—Temp.: 25° C. (Room)-Time Started: 0 s-2-Theta: 2.000°

### Example 8

### Crystals of Cpd. A in Zn-Salt (1:2) Form

a) Process for making the Crystalline Form of the 1:2 Zinc Salt of Cpd. A (1 equivalent Zn: 2 equivalents Cpd. A):

In a 1000 mL 3-necked flask with mechanical stirrer, 3.5 g of Cpd. A and 540 mL water are added. This mixture is heated to about 90° C. until everything goes into solution. To this clear solution a solution of 811 mg Zinc chloride in 10 mL water is added. A white precipitate appears and the mixture is then cooled to room temperature and stirred over night. The precipitated Zn salt is then isolated by filtration, washed with cold water and dried in a vacuum oven at 60° C. over night. This the Zn salt with a composition corresponding to a dihydrate. m.p. >230° C. The Zn-content of the salt is about 9.0% (theory 9.3%).

Here the Zinc salt of Cpd. A has a stoichiometry of one Zinc and two Cpd. A molecules.

b) The X-ray diffractogram of Zn-salt of Cpd. A as obtainable under a) yields the following Peaks (see also FIG. 3):

TABLE c
Powder X-Ray Diffraction Peaks for the Crystalline
Form of the 1:2 Zinc Salt of Cpd. A:

| ° deg 2⊖ | d-spacing (Å) | Relative intensity (%) |
|---|---|---|
| 6.7 | 13.14 | high |
| 9.5 | 9.28 | low |
| 12.5 | 7.08 | Medium |
| 17.7 | 5.01 | Low |
| 27.3 | 3.26 | Medium |

Type: 2Th locked—Start: 2.000°-End: 40.030°—Step: 0.017°-Step time: 107.s—Temp.: 25° C. (Room)-Time Started: 0 s-2-Theta: 2.000°.

### Example 9

### Crystals of Cpd. A in Mg-Salt (1:2) Form

a) Process for making the Crystalline Form of the 1:2 Magnesium Salt of Cpd. A (1 equivalent Mg: 2 equivalents Cpd. A):

In a 20 mL vial with magnetic stirrer, 74.20 mg of Cpd. A and 15 mL water are added. This mixture is heated to about 90° C. until everything goes into solution. To this clear solution a solution of 11.8 mg Magnesium chloride in 24 mL water is added. A white precipitate appears and the mixture is then cooled to room temperature and stirred over night. The precipitated Mg salt is then isolated by centrifugation and dried in a vacuum oven at 40° C. over night. This gives the Mg salt of a composition corresponding to a dihydrate; m.p. >230° C. The Mg-content of the salt is about 3.4% (theory 3.7%)

Here the Magnesium salt of Cpd. A has a stoichiometry of one Magnesium and two Cpd. A molecules.

b) The X-ray diffractogram of the Mg-salt of Cpd. A as obtainable under a) yields the following Peaks (see also FIG. 4):

TABLE d
Powder X-Ray Diffraction Peaks for the Crystalline
Form of the 1:2 Magnesium Salt of BCpd. A

| ° deg 2⊖ | d-spacing (Å) | Relative intensity (%) |
|---|---|---|
| 6.7 | 13.15 | high |
| 12.5 | 7.09 | low |
| 20.0 | 4.43 | low |
| 27.3 | 3.27 | low |

Type: 2Th alone—Start: 2.000°-End: 40.030°—Step: 0.017°-Step time: 0.3 s—Temp.: 25° C. (Room)-Time Started: 0 s-2-Theta: 2.000°

1. A depot formulation, this term including an implant, comprising a poorly water soluble salt of a bisphosphonate compound of formula I: 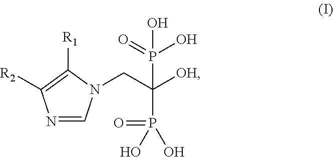
wherein one of R₁ and R₂ is hydrogen and the other is C₁-C₅-alkyl that is branched or unbranched in the form of a poorly water-soluble salt; and a polymer matrix.

2. A depot formulation of claim 1 in the form of microparticles.

3. A depot formulation according to claim 1 wherein the compound of the formula I is [2-(5-ethyl-imidazol-1-yl)-1-hydroxy-1-phosphono-ethyl]-phosphonic acid.

4. A depot formulation according to claim 1 where the poorly water soluble salt is a zinc, a magnesium or a calcium salt.

5. A depot formulation according to claim 1, where the polymer matrix comprises a linear or branched polylactide-co-glycolide.

6. A depot formulation according to claim 5, further comprising a surfactant, a porosity influencing agent and/or a basic salt.

7. A pharmaceutical composition comprising a depot formulation of claim 1 and a water-based vehicle comprising a wetting agent.

8. A composition according to claim 7, wherein the vehicle comprises a tonicity agent.

9. A composition according to claim 7, wherein the vehicle comprises a viscosity increasing agent.

10. A kit comprising a depot formulation according to claim 1 and a water-based vehicle.

11. Microparticles as such as mentioned in claim 2.

12. A poorly water-soluble salt of a compound of the formula I, 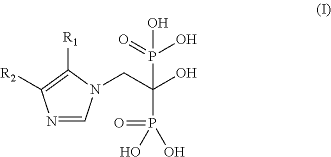
wherein one of R₁ and R₂ is hydrogen and the other is C₁-C₅-alkyl that is branched or unbranched in the form of a poorly water-soluble salt, in free or solvate form.

13. A salt according to claim 12 of [2-(5-ethyl-imidazol-1-yl)-1-hydroxy-1-phosphono-ethyl]-phosphonic acid of the formula I which is the calcium salt, where the stoichiometry of Ca: compound of formula I is 1:2

14. A crystalline form of a compound of the formula I 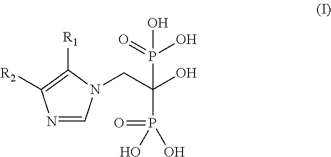
wherein one of R₁ and R₂ is hydrogen and the other is C₁-C₅-alkyl that is branched or unbranched in free form or in the form of a poorly water-soluble salt, in free or solvate form, selected from the group of crystal forms defined as follows: a crystalline form of the free zwitterionic form of [2-(5-ethyl-imidazol-1-yl)-1-hydroxy-1-phosphono-ethyl]-phosphonic acid, which has an X-ray powder diffraction pattern with at least one, two, three, or all of the following peaks at an angle of refraction 2 theta (θ) of 10.5, 13.1, 14.7, 17.2, 23.5, 25.2, 34.4, each±0.2; alternatively, at least 80% by weight of compound A in the free zwitterionic form shows such X-ray powder diffraction pattern;
a crystalline form of the calcium salt of [2-(5-ethyl-imidazol-1-yl)-1-hydroxy-1-phosphono-ethyl]-phosphonic acid with a stoichiometry of one calcium and two molecules of compound A, which has an X-ray powder diffraction pattern with at least one, two, three, or all of the following peaks at an angle of refraction 2 theta (θ) of 7.9, 10.6, 12.1, 25.7, 27.4, 29.2, each±0.2; alternatively, at least 80% by weight of the calcium 1:2 salt of compound A shows such X-ray powder diffraction pattern;
a crystalline form of the zinc salt of [2-(5-ethyl-imidazol-1-yl)-1-hydroxy-1-phosphono-ethyl]-phosphonic acid with a stoichiometry of one zinc and two molecules of compound A, which has an X-ray powder diffraction pattern with at least one, two, three, or all of the following peaks at an angle of refraction 2 theta (θ) of 6.7, 9.5, 12.5, 17.7, 27.3, each±0.2; alternatively, at least 80% by weight of the zinc 1:2 salt of compound A shows such X-ray powder diffraction pattern; and
a crystalline form of the magnesium salt of [2-(5-ethyl-imidazol-1-yl)-1-hydroxy-1-phosphono-ethyl]-phosphonic acid with a stoichiometry of one magnesium and two molecules of compound A, which has an X-ray powder diffraction pattern with at least one, two, three, or all of the following peaks at an angle of refraction 2 theta (θ) of 6.7, 12.5, 20.0, 27.3, each±0.2; alternatively, at least 80% by weight of the magnesium 1:2 salt of compound A shows such X-ray powder diffraction pattern.

15. A method of treatment and prevention of a disease or disorder where abnormal bone turnover is found, comprising administering a depot formulation according to claim 1, a composition according to claim 7, a kit according to claim 10, microparticles according to claim 12, or a crystal form according to claim 14 to a patient in need of such treatment in a therapeutically effective dosage.

16. A pharmaceutical composition according to claim 7, wherein the wetting agent is a poloxamer or a polyoxyethylene-sorbitan-fatty acid ester.

17. A poorly water-soluble salt of a compound of the formula I, according to claim 12, wherein the poorly water-soluble salt is a zinc, magnesium or a calcium salt.
